(19) United States
(12) Patent Application Publication
(10) Pub. No.: US 2008/0014285 A1
(43) Pub. Date: Jan. 17, 2008

(54) METHOD OF TREATING NEURODEGENERATIVE BRAIN DISEASE WITH A COMPOSITE COMPRISING SUPERPARAMAGNETIC NANOPARTICLES AND A THERAPEUTIC COMPOUND

(76) Inventors: Thomas M. Di Mauro, Southboro, MA (US); Sean Lilienfeld, Sharon, MA (US); Mohamed Attawia, Canton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/457,358

(22) Filed: Jul. 13, 2006

(51) Int. Cl. A61K 33/24; A61K 33/06

(52) U.S. Cl. 424/617; 424/682; 977/906

(57) ABSTRACT

A method of treating brain injury involving intrathecally administering a composite powder comprising superparamagentic nanoparticle and a therapeutic agent compound, and then magnetically transporting the composite into an injured brain.

### BACKGROUND OF THE INVENTION

In the treatment of many diseases of the nervous system, such as Alzheimer's Disease, Parkinson's Disease, brain injury and Multiple Sclerosis, therapeutic agents are often developed that show significant efficacy and safety when injected intracranially into the patient. However, many of these same therapeutics are not able to cross the blood brain barrier and so are not amenable to administration by a relatively minimally invasive means.

U.S. Pat. Nos. 4,869,247 (“Howard I”) and, U.S. Pat. No. 6,216,030 (“Howard II”) disclose drilling a burr hole in the cranium, depositing a magnetic implant into the hole, and then magnetically driving the implant to a desired location in the cranium. These patents disclose that the magnetic implant may be a carrier for therapeutic drugs.

The literature has provided application of the Howard I and II patents through Howard, Neurosurgery, March 1989, 24(3) 444-8 (“Howard III”) and Grady, Med. Phys., May-June 1990 17(3) 405-15 (“Grady”). However, Howard III and Grady respectively disclose the use of 3 mm diameter and 5 mm diameter magnetic implants. These relatively large implants are moved only very slowly by magnetic fields, and are limited in the amount of drug they can deliver.

### SUMMARY OF THE INVENTION

The present inventors have developed inventions for treating brain injury with a composite comprising a superparamagnetic nanoparticle compound and various therapeutic compounds.

The nanoparticle size of the superparamagnetic compound provides advantage over the millimeter sized bodies disclosed by Howard. First, the very high surface area to weight ratio of such nanoparticles increase the amount of therapeutic agent or carrier that can be coated onto the nanoparticle per gram of carrier. Accordingly, more therapeutic agent can be delivered to the patient for the same weight of superparamagnetic compound. Second, the nanoparticulate size helps in the quick bioabsorption of the superparanagnetic compound, thereby avoiding the need to remove the particle and allowing for sustained delivery of the therapeutic agent.

In some embodiments, the composite is administered intrathecally (such as through a lumbar puncture) and then magnetically guided through the spinal CSF and into the cranium to the site of the brain defect. Once sited at the location of the injury, the therapeutic compound is released from the composite and ameliorates the brain injury.

Therefore, in accordance with the present invention, there is provided a method of treating brain injury comprising:
a) intrathecally administering a composite powder comprising superparamagentic nanoparticle and a therapeutic agent compound, and
b) magnetically transporting the composite into an injured brain.

In some embodiments, the blood brain barrier of the patient has been disrupted, either as part of the disease state, as in the case of stroke or AD, or intentionally, such as through the administration of mannitol. In these situations, the composite comprising a superparamagnetic nanoparticle compound and various therapeutic compounds may be administered intravenously. Subsequent application of a magnetic field upon the brain acts to localize such intravenously injected composites in the brain and may also cause extended retention of the composite in the brain.

### DETAILED DESCRIPTION OF THE INVENTION

In some embodiments, the superparamagnetic compound comprises iron, and is preferably iron oxide. In some embodiments, the iron oxide is in the form of superparamagnetic iron oxide nanoparticles (SPION). As SPION particles have been proposed for use as a brain contrast agent (Kent, Magn., Resonance Med., March 1990 13(3), 434-43) and Kicher, Can. Res. 63, 2003, 8122-5 (Kicher I), it is understood that they are biocompatible. Some exemplary SPION particles are described in Kircher, Bioconj. Chem., March-April 2004 15(2) 242-8 (Kircher II) and Kircher, Mol. Imaging, April-June 2002 1(2) 89-95 (Kicher III).

In some embodiments, the superparamagnetic compound comprises manganese, and is preferably magnesium-doped hydroxyapatite (Mn-HA). Ayhan, Plast. Reconstr. Surg., Apr. 1, 2002, 109(4) 1333-7 studied the bioabsorbable osseous fixation materials in the brain, and found that they have a negligible effect upon brain tissue.

In some embodiments, the superparamagnetic compound comprises gadolinium.

Preferably, the diameter of the superparamagnetic nanoparticle is between about 10 nm and about 1000 nm, preferably between 30 nm and 300 nm, more preferably between 50 nm and 200 nm.

The therapeutic agents that may be beneficially delivered to the brain include but are not limited to magnesium compounds, anti-excitotoxic compounds (such as lubeluzole), neurotrophins, growth factors, agents that bind to beta amyloid protein with high affinity, and anti-inflammatory compounds.

The composites of the present invention may be used to treat neurodegenerative diseases such as Alzheimer's Disease, Parkinson's Disease, traumatic brain injury (TBI) and Multiple Sclerosis.

According to Vink, Exp. Op. Invest. Drugs, October 2002, 11(1) 1375-86, and Vink, Exp. Op. Invest. Drugs, (2004) 13(10) 1263-74, “traumatic brain injury (TBI) is one of the leading causes of death and disability in the industrialized world and remains a major health problem with serious socioeconomic consequences. In industrialized countries, the mean per capita incidence of traumatic brain injury (TBI) that results in a hospital presentation is 250 per 100,000. In Europe and North America alone, this translates to more than 2 million TBI presentations annually. Approximately 25% of these presentations are admitted for hospitalization. Those individuals who survive TBI are often left with permanent neurological deficits, which adversely affect the quality of life and as a result, the social and economic cost of TBI is substantial. Despite the significance of these figures, there is no single interventional pharmacotherapy that has shown efficacy in the treatment of clinical TBI).”

According to Vink, Exp. Op. invest. Drugs, (2004) 13(10) 1263-74, “brain magnesium decline is a ubiquitous feature of TBI and is associated with the development of neurological deficits. Experimentally, parental administration of magnesium no more than 12 hours post-trauma restores brain magnesium homeostasis and profoundly improves both motor and cognitive outcome. While the mechanism of action is unclear, magnesium has been shown to attenuate a variety of secondary injury factors such as brain edema, cerebral vasopspasms, glutamate excitotoxicity, calcium-mediated events, lipid peroxidation MPT and apoptosis.”

Despite the therapeutic properties of magnesium , the delivery of magnesium to the affect brain tissue remains an issue. For example, Brewer, Clin. Neuropharmacol., November-December 2001 24(6) 341-5 reported that systemic administration of magnesium sulfate failed to increase CSF ionized magnesium concentration in patients with intracranial hypertension despite increasing plasma magnesium levels by >50%. McKee, Crit. Care Med., March 2005 33(3) 661-6 investigated the brain bioavailability of peripherally administered magnesium sulfate, and reported that such hypernagnesia produced only marginal increases in total and ionized CSF [Mg]. McKee concluded that regulation of CSF [Mg] is largely maintained following acute brain injury and limits brain bioavailability of magnesium sulfate.

The present inventors have developed inventions for treating brain injury with a composite comprising a superparamagnetic compound (preferably in nanoparticle form) and a magnesium compound.

In preferred embodiments, the composite in administered intrathecally (such as through a lumbar puncture) and then magnetically guided through the spinal CSF and into the cranium to the site of the brain injury. Once sited at the location of the injury, the magnesium is released from the composite and ameliorates the brain injury.

Therefore, in accordance with the present invention, there is provided a method of treating brain injury comprising:
a) intrathecally administering a composite powder comprising superparamagnetic compound and a magnesium compound, and
b) magnetically transporting the composite into an injured brain.

The magnesium compound can be selected from the group consisting of magnesium metal, magnesium oxide, magnesium stearate, magnesium citrate, magnesium chloride, magnesium sulfate, magnesium carbonate, magnesium hydroxide, magnesium gluconate, magnesium phosphate and magnesium aspartate.

When MgO is selected as the magnesium compound, it is preferably provided in the form of a powder, as MgO is more readily hydrolysable when in a powder form. When MgO is hydrolyzed, it forms Mg(OH)₂, which solubilizes to Mg⁺² cations and OH⁻ anions. Since it is known that, during brain injury, the pH of brain tissue decreases (Gupta, J. Neurotrauma, June 2004 21(6) 678-84), the release of hydroxide ions from magnesium hydroxide beneficially restores the pH of brain tissue.

In some embodiments, the magnesium oxide is provided as nanorods. In preferred embodiments thereof, the composite takes the form of iron oxide nanotubes overlying MgO nanorods. Such composites are described in Liu, J. Am. Chem. Soc., 2005, 127(1), 6-7, the text of which is incorporated by reference herein. In use, the open end of the MgO nanorod is gradually etched by the cerebrospinal spinal fluid (CSF), thereby forming Mg⁺² cations and OH⁻ anions.

In some embodiments, the aspect ratio of such nanorod: nanotube composite particluates is less than 30(length):1(diameter), preferably less than 10:1, more preferably less than 3:1. Because dissolution of the MgO occurs via hydrolysis through the end faces of the nanorods, in general, the greater the aspect ratio of the particulate, the longer the time required for the MgO contained therein to dissolve. Foe example, Liu reports that the MgO inner cores of the MgO/Fe₃O₄ core-shell nanowires were selectively etched in (NH₄)₂SO₄ (Alfa Aesar, 99.99%) solution (10 wt %, pH≈6.0) at a temperature of 80° C., and that an etching time of about 1.5 h was typically used to obtain micrometer-long, 30 nm diameter Fe₃O₄ nanotubes with completely etched inner cores. Thus, desiging aspect ratios to be much less than 30:1 is desirable.

The length of the particulate is preferably between 10 nm and 10 um, more preferably between 30 nm and 1 um, more preferably between 50 nm and 300 nm.

The thickness of the iron oxide nanotube is preferably at least 10 nm, preferably at least 30 nm, more preferably at least 50 nm. In general, the thicker the iron oxide coating, the more susceptible the particulate to magnetic navigation.

Adjustment of the composite length and diameter, and iron oxide coating thickness is carried out in accordance with Han, S.; Li, C.; Liu, Z.; Lei, B.; Zhang, D.; Jin, W.; Liu, X.; Tang, T. Nano Lett. 2004, 4, 1241, and Zhang, D.; Liu, Z.; Hoan, S.; Li, C.; Lei, B.; Stewart M. P.; Tour, J. M.; Zhou, C. Nano Lett. 2004, 4, 2151.

In some embodiments, the magnesia (MgO) is provided in a nanoparticle composite comprising iron oxide and magnesia. In some embodiments, the composite disclosed in Moscovici, J. Syncronization Radiat., March 1, 2001 8(Pt.2), 925-7, is selected. Moscovici describes coating an MgO nanoparticle with FeO, resulting in a composite wherein the Fe is mixed into the MgO phase.

The diameter of the nanoparticulate is preferably between 10 nm and 10 um, more preferably between 30 nm and 1 um, more preferably between 50 nm and 300 nm.

In some embodiments, the MgO is provided as a nanoparticulate layer upon an iron oxide nanoparticle. Preferably, the MgO is coated upon the iron oxide surface by first coating the iron oxide surface with Mg(NO₃)₂.H₂O, ad then calcining the composite at 500° C. to produce an MgO layer. In some embodiments, calcination is carried out in accordance with Lin, J. Mat. Sci. Mater. Med., January 2005 16(1), 53-6.

In some embodiments, the MgO is provided as a co-precipitated powder with iron oxide.

In some embodiments, magnesium hydroxide (Mg(OH)₂ is selected as the magnesium compound. Wen Mg(OH)₂ contacts water, it solubilizes to Mg⁺² cations and OH⁻ anions. Since it is known that, during brain injury, the pH of brain tissue decreases (Gupta, J. Neurotrauma, June 2004 21(6) 678-84), the release of hydroxide ions from magnesium hydroxide beneficially restores the pH of brain tissue.

In some embodiments wherein Mg(OH)₂ is selected as the magnesium compound, it is provided in a PLGA carrier in accordance with the techniques described in Aubert-Pouessel, Brain Res. July 2002, 19,7,1046-51

In some embodiments, MgCl₂ is selected as the magnesium compound. In some preferred embodiments thereof, MgCl₂ is loaded into a polyethylene vinyl acetate (PEVAc) carrier in accordance with the techniques disclosed in Vasudev, Drug Delivery, 6, 1147-126 (1999).

In some preferred embodiments thereof, MgCl₂ is loaded into a PLGA carrier in accordance with the techniques disclosed in Shenderova, Pharm. Res., February 16(2) 241-8.

In some preferred embodiments thereof, MgCl₂ is loaded into a dextran-PEG 400 carrier in accordance with the techniques disclosed in Bronsted, J. Controll. Rel., 53 (1998) 7-13.

In some embodiments, MgCO₃ is selected as the magnesium compound. In preferred embodiments thereof, MgCO₃ is loaded into a PLGA carrier in accordance with the techniques disclosed in Sandor, Biochim. Biophys. Acta., Feb. 15, 2002, 1570(1) 63-74.

In addition to TBI, it is believed that the methods and devices of the present invention could be useful in increasing the magnesium level in the brain of a stroke patient, or in the brain of an epileptic patient, or in the brain of a patient having Parkinson's Disease (PD). Muir, Postgrad Med. J. 202, 78, 641-645 reports that systemically administered magnesium is useful in treating stroke.

According to Marranness, J. Pharmacol. Exp. Therapeutics, 295(2) 2000, 531-545, lubeluzole is the (+)−S enantiomer of a benzothiazole derivative that has a neuroprotective action in animal models of focal and global ischemia, in which it reduces sensormotor deficits and the infarct volume, Lubeluzole inhibits glutamate-induced nitric oxide related neurotoxicity and blocks nieurotoxicity induced by nitric oxide donors. Because of these qualities, lubeluzole has been proposed as a therapeutic in early stage ischemic stroke. However, maintenance of adequate CNS levels of lubeluzole has been found to be problematic.

The present inventors have developed inventions for treating brain injury and stroke with a composite comprising a superparamagnetic compound and a thiazole-containing compound.

In some embodiments, the composite in administered intrathecally (such as through a lumbar puncture) and then magnetically guided through the spinal CSF and into the cranium to the site of the brain injury. Once sited at the location of the injury, the thiazole-containing compound is released from the composite and ameliorates the stroke.

Therefore, in accordance with the present invention, there is provided method of treating stroke in a brain of a patient, comprising:
administering a composite powder comprising superparamagentic nanoparticle and an effective amount of a thiazole, and
imposing a magnetic field upon the brain.

In some embodiments, the blood brain barrier of the patient has been disrupted as part of the stroke disease state or intentionally, such as through the administration of mannitol. In these situations, the composite comprising a superparamagnetic nanoparticle compound and lubeluzole may be administered intravenously. A magnetic field from an external magnet is then impressed upon the infarcted portion of the brain in order to localize the composite in that region.

It is understood by the present inventors that the short residence time of lueluzole in the brain may be responsible for a decrease in the effectiveness of its simple intravenous administration in stroke. Without wishing to be tied to a theory, it is believed that the imposing a magnetic field upon the brain will increase the residence time of the composite comprising superparamagnetic nanoparticles in the brain, and thereby cause an increase in the concentration of lubeluzole at the target site and prolong the residence time of lubeluzole in the brain, thereby obviating maintenance dosing requirements.

Preferably, the thiazole-containing compound is a benzothiazole, more preferably lubeluzole.

In some embodiments, lubeluzole is delivered to the brain via in a carrier comprising PEG. PEG appears to be the primary ingredient of the oral drops of Example 12 of U.S. Pat. No. 5,434,168 (“the lubeluzole patent”), the specification of which is incorporated by reference in its entirety.

Briefly, 50 g of lubeluzole. is dissolved in 0.5 liters of 2-hydroxypropanoic acid and 1.5 liters of the polyethylene glycol (PEG) at about 60° C.-80° C. After cooling to about 30° C.-40° C., there are added about 35 liters of polyethylene glycol and the mixture is stirred well. Polyethylene glycol is then added to a volume of 50 liters providing a solution comprising 1 mg/ml of lubeluzole. The resulting solution is filled into suitable containers.

In some embodiments, lubeluzole is delivered to the brain via a cellulose carrier. Cellulose is frequently cited as a earner for benzothiazole derivatives, and is listed as an ingredient in the tablet example 15 of the lubeluzole patent.

The present inventors have further developed inventions for treating neurodegenerative disease with a composite comprising a superparamagnetic compound and a neurotrophin.

In preferred embodiments, the composite in administered intrathecally (such as through a lumbar puncture) and then magnetically guided through the spinal CSF and into the cranium to the site of the brain injury. Once sited at the location of the injury, the neurotrophin is released from the composite and ameliorates the disease.

In some embodiments, the neurotrophin is NGF. When NGF is selected as the neurotrophin, it may be combined with a PLGA carrier. Techniques for providing NGF in a PLGA carrier are disclosed in Camarata, Neurosurg., Mar. 30, 1992, 3,313-319, and in Hadlock, J. Reconstr. Microsurg., April 2003, 19(3), 179-84, 185-6.

In other embodiments, NGF may be directly coated upon iron oxide.

In some embodiments, the neurotrophin is BDNF. When BDNF is selected as the neurotrophin, it may be combined with a PLGA carrier. Techniques for providing BDNF in a PLGA carrier are disclosed in Mittal, Neuroreport., Dec. 20, 1994, 5, 18,2577-82.

In some embodiments, the neurotrophin is CTNF. When CTNF is selected as the neurotrophin, it may be combined with a PLGA carrier. Techniques for providing CTNF in a PLGA carrier are disclosed in Maysinger, Exp. Neurol., April 1996, 138(2) 177-188.

In some embodiments, the neurotrophin is GDNF. When GDNF is selected as the neurotrophin, it may be combined with a PLGA carrier. Techniques for providing GDNF in a PLGA carrier are disclosed in Aubert-Pouessel, J. Controll. Release, Mar. 24, 2004 95,3,463-475.

The present inventors have fiurther developed inventions for treating neurodegenerative disease with a composite comprising a superparamagnetic compound and a growth factor.

In preferred embodiments, the composite in administered intrathecally (such as through a lumbar puncture) and then magnetically guided through the spinal CSF and into the cranium to the site of the brain injury. Once sited at the location of the injury, the growth factor is released from the composite and ameliorates the disease.

In some embodiments, the growth factor is IGF-I. When IGF-I is selected as the growth factor, it may be combined with a PLGA carrier. Techniques for providing IGF-I in a PLGA carrier are disclosed in Carrascosa, Biomaterials, Feb. 25, 2004, 4, 707-714.

In some embodiments, the growth factor is VEGF. The literature reports that VEGF can he neuroprotective in both Alzheimer's Disease (AD) and Parkinson's Disease (PD). Yang, J. Neurochem., April 2005, 93(1) 118-127 reports that VEGF provides neuroprotection in AD by binding to beta amyloid protein. Yasuhara, Brain Research, Mar. 15, 2005, 1038, 1, 1-10 reports that low dose VEGF is neuroprotective towards dopaminergic neurons in PD.

When VEGF is selected as the growth factor, it may be combined with a PLGA carrier. Techniques for providing VEGF in a PLGA carrier are disclosed in Faranesh, Magn. Reson. Med., June 2004, 51,6,1265-1271.

In some embodiments, the growth factor is GDF-5. When GDF-5 is selected as the growth factor, it may be combined with a PLGA carrier.

In some embodiments, the anti-tumor therapeutic is BCNU. When BCNU is selected as the anti-tumor therapeutic, is may be combined with a PLGA carrier. Techniques for providing BCNU in a PLGA carrier are disclosed in Chae, Int. J. Pharm., Jul 14, 2005(e-pub).

The present inventors have further developed inventions for treating neurodegenerative disease with a composite comprising a superparamagetic compound and a compound having a high affinity for soluble beta amyloid protein.

In preferred embodiments, the composite in administered intrathecally (such as through a lumbar puncture) and then magnetically guided through the spinal CSF and into the cranium to the site of the brain injury. Once sited at the location of the injury, the compound having a high affinity for soluble beta amyloid protein is released from the composite and ameliorates the disease.

In some embodiments, the compound having a high affinity for soluble beta amyloid protein is VEGF. Yang, J. Neurochem., April 2005, 93(1) 118-127 reports that VEGF provides neuroprotection in AD by binding to beta amyloid protein.

When VFGF is selected as the compound having a high affinity for soluble beta amyloid protein is VEGF, it may be delivered in accordance with Faranesh, Magn. Reson. Med, June 2004, 51(6), 1265-71, which describes the production of a supermagnetic composite comprising Gadolimium and VEGF in 48 micron PLGA microspheres. VECE was loaded at 163 ng/mg polymer. Gadolinium was loaded at 17 ug/mg polymer

In addition to VEGF, there are other molecules that have high affinity binding to beta amyloid protein. These include gelsolin and GM1 (Matsuoka, J. Neurosci., Jan. 1, 2003, 23(1) 29-33), and Congo Red, Chrysamine, and Thiflavin S (Lee, Neurobiol. Aging, November December 2002, 23(6) 1039-1042).

In some embodiments, magnetic Mn-HA particles are coated with VEGF and then injected intrathecally (such as through a lumbar puncture). Magnetic forces are then used to transport them to the brain, wherein VEGF binds to the beta amyloid proteins in the CSF of the AD patient. Removal of the BAP-VEGF-Mn-HA conjugate a few weeks later can likewise be driven by magnetic forces.

The present inventors have further developed inventions for treating neurodegenerative disease with a composite comprising a superpararnagnetic compound and an anti-inflammatory compound.

In preferred embodiments, the composite in administered intrathecally (such as through a lumbar puncture) and then magnetically guided through the spinal CSF and into the cranium to the site of the brain injury. Once sited at the location of the injury, the anti-inflamnmatory compound is released from the composite and ameliorates the disease.

In some embodiments, the anti-inflammatory compound is an antagonist is capable of specifically inhibiting a pro-inflammatory cytokine (“HSCA”). In some embodiments, the antagonist is capable of specifically inhibiting a pro-inflammatory cytokine selected from the group consisting of TNF-α an interleukin (preferably, IL-1, Il-6 and IL-8), FAS, an FAS ligand, and IFN-gamma. In some embodiments, the HSCA inhibits the cytokine by preventing its production. In some embodiments, the HSCA inhibits the cytokine by binding to a membrane-bound cytokine. In others, the HSCA inhibits the cytokine by binding to a solubilized cytokine. In some embodiments, the HSCA inhibitor inhibits the cytokine by both binding to membrane bound cytokines and to solubilized cytokine. In some embodiments, the HSCA is a monoclonal antibody (“mAb”). The use of mAbs is highly desirable since they bind specifically to a certain target protein and to no other proteins. In some embodiments, the HSCA inhibits the cytokine by binding to a natural receptor of the target cytokine.

In some embodiments, the HSCA inhibits the cytokine by preventing its production. One example thereof is an inhibitor of p38 MAP kinase. In some embodiments, the TNF inhibitor inhibits the TNF by binding to membrane bound TNF in order to prevent its release from membrane. In others, the TNF inhibitor inhibits the TNF by binding to solubilized TNF. One example thereof is etanercept. In some embodiments, the TNF inhibitor inhibits the TNF by both binding to membrane bound TNF and to solubilized TNF. One example thereof is infliximab. In some embodiments, the HSCA inhibits the cytokine by binding to a natural receptor of the target cytokine.

Preferred TNF antagonists include, but are not limited to the following.: etanercept (Enbrel®-Amgen); infliximab (Remicade®-Johnson and Johnson); D2E7, a human anti-TNF monoclonal antibody (Knoll Pharrnaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R1) (Amgen); and onercept, a recombinant TNF binding protein (r-TBP-1) (Serono).

TNF antagonists suitable for compositions, combination therapy, co-administration, devices and/or methods of the present invention (further comprising at least one anti body, specified portion and variant thereof, of the present invention), include, but are not limited to, anti-TNF antibodies (e.g., at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, enteracept (Enbrel™), adalimulab (Humira™), CDP-571, CDP-870, afelimomab, lenercept, and the like), antigen-binding fragments thereof and receptor molecules which bind specifically to TNF; compounds which prevent and/or inhibit TNF synthesis, TNF release or its action on target cells, such as thalidomide, tenidap, phosphodiesterase inhibitors (e.g, pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor antibody,” “BF antibody,” “TNFα antibody,” or fragment and the like decreases, blocks, inhibits, abrogates or interferes with TNFα activity in vitro, in situ and/or preferably in vivo. For example, a suitable TNF human antibody of the present invention can bind TNFα and includes anti-TNF antibodies, antigen-binding fragments thereof, and specified mutants or domains thereof that bind specifically to TNFα. A suitable TNF antibody or fragment can also decrease block, abrogate, interfere, prevent and/or inhibit TNF RNA, DNA or protein synthesis, TNE release, TNF receptor signaling, membrane TNF cleavage, TNF activity, TNF production and/or synthesis.

Chimeric antibody cA2 consists of the antigen binding variable region of the high-specificity neutralizing mouse anti-human TNFα IgG1 antibody, designated A2, and the constant regions of a human IgG1, kappa immunoglobulin. The human IgG1 Fc region improves allogeneic antibody effector function, increases the circulating serum half-life and decreases the immunogenicity of the antibody. The avidity and epitope specificity of the chimeric antibody cA2 is derived from the variable region of the murine antibody A2. In a particular embodiment, a preferred source for nucleic acids encoding the variable region of the murine antibody A2 is the A2 hybridoma cell line.

Chimeric A2 (cA2) neutralizes the cytotoxic effict of both natural and recombinant human TNFα in a dose dependent manner. From binding assays of chimeric antibody cA2 and recombinant human TNFα, the specificity constant of chimeric antibody cA2 was calculated to be 1.04×10¹⁰M⁻¹. Preferred methods for determining monoclonal antibody specificity and specificity by competitive inhibition can be found in Harlow, et al., antibodies: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, New York, (1992-2000); Kozbor et al., Immunol. Today, 4:72-79 (1983); Ausubel et al, eds. Current Protocols in Molecular Biology, Wiley Interscience, New York (1987-2000); and Muller, Meth. Enzymol, 92:589-601 (1983), which references are entirely incorporated herein by reference.

In a particular embodiment, murine monoclonal antibody A2 is produced by a cell line designated c134A. Chimeric antibody cA2 is produced by a cell line designated c168A.

Additional examples of monoclonal anti-TNF antibodies that can be used in the present invention are described in the art (see, e.g., U.S. Pat. No. 5,231,024; Möller, A. et al., Cytotine 2(3): 162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res, Comm. 137:847-854 (1986); Meager, et al., Hybridoma 6:305-311 (1987); Fendly et al., Hybridoma 6:359-369 (1987); Bringman, et al, Hybridoma 6:489-507 (1987); and Hirai, et at, J. Immunol Meth. 96:57-62 (1987), which references are entirely incorporated herein by reference).

Preferred TNF receptor molecules useful in the present invention are those that bind TNFα with high specificity (see, e.g., Feldmann et al., International Publication No. WO 92/07076 (published Apr. 30, 1992); Schall et al, Cell 61:361-370 (1990); and Loetscher et al., Cell 61:351-359 (1990), which references are entirely incorporated herein by reference) and optionally possess low immunogenicity. In particular, the 55 kDa (p55 TNF-R) and the 75 kDa (p75 TNF-R) TNF cell surface receptors are useful in the present invention. Truncated fonrns of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNFa inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high specificity, as well as other undefined properties, can contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein. TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et at, J. Exp. Med. 174:1483-1489 (1991); Kolls et al., Proc. Natl. Acad. Sci. USA 91:215-219 (1994); Butler et al, Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (flied May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of TNF receptor molecule refers to the portion of the TNF receptor molecule, or the portion of the TNF receptor molecule sequence which encodes TNF receptor molecule, that is of sufficient size and sequences to functionally resemble TNF receptor molecules that can be used in the present invention (e.g., bind TNFα with high specificity and possess low immunogenicity). A functional equivalent of TNF receptor molecule also includes modified TNF receptor molecules that functionally resemble TNF receptor molecules that can be used in the present invention (e.g., bind TNFα with high specificity and possess low immunogenicity). For example, a functional equivalent of TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2003).

In some embodiments, the monoclonal antibody that inhibits TNF-a is selected from the group consisting of monoclonal rodent-human antibodies, rodent antibodies, human antibodies or any portions thereof, having at least one antigen binding region of an immunoglobulin variable region, which antibody binds TNF. Preferably, this monoclonal antibody is selected from the group of compounds disclosed in U.S. Pat. No. 6,277,969, the specification of which is incorporated by reference. In some embodiments, the infliximab is delivered in a formulation having an infliximab concentration of between about 30 mg/ml and about 60 mg/ml.

In some embodiments, the specific inhibitor of TNF-a is an inhibitor of p38 MAP kinase, preferably, a small molecule inhibitor of p38 MAP kinase. The inhibition of p38 MAP kinase is believed to block production of both TNF-a and Il-2, both of which are pro-inflammatory cytokines. The small molecule inhibitors of p38 MAP kinase are very specific & potent (˜nM). Without wishing to be tied to a theory, it is believed that inhibition of p38 should not block TGF signaling nor TGF activity. It is further believed that p38 inhibitors may also block induction of some metalloproteinases, COX 2 and NO synthetase. It is further believed that P38 inhibitors do not inhibit interleukins involved in immune cell proliferation such as IL-2.

In some embodiments, the HSCA is a specific antagonist of an interleukin. Preferably, the target interleukin is selected from the group consisting IL-1, IL-2, IL-6 and IL-8, and IL-12. Preferred antagonists include hut are not limited to Kineretg (recombinant IL 1-RA, Amgen), IL1-Receptor Type 2 (Aingen) and IL-1 Trap (Regeneron).

In some embodiments, the anti-inflammatory compound comprises aMSH, IL-10 or adiponectin.

PLGA may be a carrier for aMSH. Bhardwaj, Pharm. Res., May 2000 17(5) 593-9 teaches the release of alpha-MSH from PLGA over 24 hours.

Among the therapeutic agents which may be micro encapsulated and administered into the cerebrospinal fluid according to the present invention can be, preferably, anti-inflammatory agents. As used herein the term “anti-inflammatory agents refers to any agent which possesses the ability to reduce or eliminate cerebral edema (fluid accumulation), cerebral ischemia, or cell death caused by traumatic brain injury (TBI) or stroke. Categories of anti-inflammatory agents include:
a) Free radical scavengers and antioxidants, which act to chemically alter (dismutate) or scavenge the different species of oxygen radicals produced due to ischemic and trauma associated events. Unless dismutated or scavenged, these highly reactive free radicals cause the peroxidation (breakdown) of cell membrane phospholoipids (lipid peroxidation) and the oxidation of cellular proteins and nucleic acids leading to severe tissue damage and death of neurons. Examples of such drugs are superoxide dismutase, catalase, nitric oxide, mannitol, allopurinol, dimethyl sulfoxide.
b) Nonsteroidal anti-inflammatory drugs (NSAIDS), which act to reduce cell migration, caused by ischemic and trauma associated events, and therefore slow down edema formation, as well as provide pain relief. Examples of such drugs are aspirin, acetaminophen, indomethacin, ibuprofen.
c) Steroidal anti-inflammatory agents (Glucocorticoids, Hormones), which can enhance or prevent the immune and inflammatory process and inhibit lipid peroxidation as seen in the events that occur during oxygen radical formation. Examples of such drugs are cortisone, prednisone, prednisolone, dexamethasone. The most well known of these is dextramethasone which has been used for reduction of cerebral edema after TBI.
d) Calcium channel blockers, which act to prevent excess calcium from entering the cell during cerebral ischemia. Some of these drugs also have other beneficial effects on increasing cerebral blood flow to the brain. Examples of such drugs are nimodipine, nifedipine, veraparnil, nicardipine.
e) NMDA antagonists, which block the NMDA receptor site for glutamate, a neurotransmitter released excessively during ischemia. Excess glutamate can activate the NMDA receptors leading to increase firing which will in turn cause cell swelling and an influx of calcium leading to cell death. Examples of such drugs are magnesium sulfate and dextromethorphan, actually an opioid analogue.
f) citicholine. Citicholine prevents toxic free fatty acid accumulation, promotes recovery of brain function by providing two components, cytidine and choline, required in the formation of nerve cell membrane, promoting the synthesis of acetylcholine. a neurotransmitter associated with cognitive function.
g) Heat shock proteins, such as hsp70 or hsp 27.
h) recombinant glutamate receptors, such as GluR1.

### The therapeutic agents can be used alone or in combination with one or more other therapeutic agents to achieve a desired effect.

In many embodiments, the composite preferably further includes a controlled-release carrier layer that carries and then releases the therapeutic compound. Preferably, this carrier layer is provided in the form of a bioerodable polymer.

In some embodiments, the therapeutic compound is provided in a water-soluble high molecular weight cellulose polymer. High molecular weight cellulose polymer refers to a cellulose polymer having an average molecular weight of at least about 25,000, preferably at least about 65,000, and more preferably at least about 85,000. The exact molecular weight cellulose polymer used will generally depend upon the desired release profile. For example, polymers having an average molecular weight of about 25,000 are useful in a controlled-release composition having a time release period of up to about 8 hours, while polymers having an average molecular weight of about 85,000 are useful in a controlled-release composition having a time released period of up to about 18 hours. Even higher molecular weight cellulose polymers are contemplated for use in compositions having longer release periods. For example, polymers having an average molecular weight of 180,000 or higher are useful in a controlled-release composition having a time release period of 20 hours or longer.

The controlled-release carrier layer preferably consists of a water-soluble cellulose polymer, preferably a high molecular weight cellulose polymer, selected from the group consisting of hydroxypropyl methyl cellulose (HPMC), hydroxyethyl cellulose (HEC), hydroxypropyl cellulose (HPC), carboxy methyl cellulose (CMC), and mixtures thereof. Of these, the most preferred water-soluble cellulose polymer is HPMC. Preferably the HPMC is a high molecular weight HPMC, with the specific molecular weight selected to provide the desired release profile.

The HPMC is preferably a high molecular weight HPMC, having an average molecular weight of at least about 25,000, more preferably at least about 65,000 and most preferably at least about 85,000. The HPMC preferably consists of fine particulates having a particle size such that not less than 80% of the HPMC particles pass through an 80 mesh screen. The HPMC can be included in an amount of from about 4 to about 24 wt %, preferably from about 6 to about 16 wt % and more preferably from about 8 to about 12 wt %, based upon total weight of the composition.

In preferred embodiments, the cellulose is coated upon an iron oxide core nanoparticle via the methods described in Tilcock, J. Magn. Meson. Imaging, Jul.-Aug. 1, 1991 4,463-7. In other embodiments, the cellulose is coated upon an iron oxide core nanoparticle via the methods described in U.S. Pat. No., 6,207,134, the specification of which is incorporated by reference in its entirety. See Example 12 in particular.

The brain biocompatibility of cellulose-based materials has been reported in the literature. See Tamargo, J. Biomed. Mater. Res., February 1989 23(2) 253-66, and Menei, J. Biomed. Mater. Res., September 1994 28(9), 1079-8.5.

In some embodiments, the therapeutic compound is provided in PLGA. Techniques for coating superparamagnetic nanoparticles in PLGA are provided in Faranesh, Magn. Reson. Med., June 2004 51,6,1265-1272, and in Carino, J. Controll. Release, Mar. 1, 2000, 65(1-2), 261-9.

In some embodiments, PLGA microbubbles are the carrier and are prepared by a double emulsion (W/O/W) solvent evaporation process.

In preferred embodiments thereof, the double emulsion is produced by following the teachings of El-Sherif, J. Biomed. Mat Res., 66A:347-55, 2003. In particular 0.5 g of PLGA is dissolved in 20 mL of methylene chloride. To generate the first W/O emulsion, 1.0 mL of deionized water is added to the polymer solution and probe sonicated at 110 W for 30 seconds. The W/O emulsion is then poured into a 5% PVA solution and homogenized for 5 minutes at 9500 rpm. The PVA acts as a surfactant and reduces the surface tension, whereas simultaneous homogenization breaks the W/O emulsion into a population of small beads. The double (W/O)/W emulsion is then poured into a 2% isopropanol solution and stirred at room temperature for 2 hours to evaporate off the methylene chloride and thus harden the capsules. The capsules are then collected by centrifugation, washed one time with deionized water, centrifuiged at 15° C. for 5 minutes at 5000 g, and the supernatant is discarded. The capsules are then washed three times with hexane to further extract the methylene chloride from the polymer beads. The capsules are then frozen in a −85° C. freezer and lyophilized using a freeze dryer to fully dry the capsules and sublime the encapsulated water.

In some embodiments, the therapeutic agent is encapsulated by the polymer matrix. In these embodiments, the therapeutic agent is added after the PLGA is dissolved in the methylene chloride and before addition of the PVA to make the double emulsion.

In some embodiments, the superparamagnetic compound and the therapeutic agent are added to the deionized water that is used to generate the first W/O emulsion, This results in the the superparamagnetic compound and the therapeutic agent being encapsulated in the carrier (e.g., PLGA) polymer.

In some embodiments, the therapeutic compound is provided in polyethylene glycol (PEG). PEG may be provided. as a coating upon SPION in accordance with Gupta, IEEE Trans. Neurobioscience, March 2004, 3(1) 66-73, or Gupta, J. Mat. Sci. Mater. Med., April 2004, 15(4) 493-6.

In some embodiments, the cartier layer comprises poly(ethylene vinyl acetate)(PEVAc). Preferably, when the therapeutic compound is magnesium, the Mg-PEVAc mixture is made in accordance with Vasudev, Drug Delivery, 6:117-126, 1999. Briefly, a known amount of MgCl₂ is dissolved in a 15% solution of PEVAc in chloroform at room temperature. The solution is then spread over a glass plate separated by shims and the solvent is slowly evaporated.

The cerebral use of ethylene vinyl acetate compounds as drug carriers has been investigated by Wiranowska, J. Interferon Cytokine Res., June 1998, 18(6) 377-85; Saltzman, Pharm. Res., February 1999, 16(2) 232-40; Tornqvist, Exp. Neurology, 164, 130-138(2000); Pradilla, J. Neurosurgery, July 2004, 101(1) 99-103.

In some embodiments, the composite powder is delivered to the patient intrathecally, and then guided to the infarct by magnetic means. Engelhard, Cancer Biochem. Biophys., October 1992, 13 (1), 1-12 reports the rapid movement of iron oxide inicroparticles in response to a magnetic field. Engelhard further reported that iron oxide microparticles injected into the intrathecal space could be transported through the CSF and localized to the medial aspect of a cerebral hemisphere.

In some embodiments, the blood brain barrier of the patient has been disrupted, either as part of the disease state, as in the case of stroke or AD, or intentionally, such as through the administration of mannitol. In these situations, the composite comprising a superparamagnetic nanoparticle compound and various therapeutic compounds may be administered intravenously.

In some embodiment, magnetic fields are used to intracranially drive the composite of the present invention. In some embodiment, the composite is first driven intrathecally ftom the spinal column into the cranium. In other embodiments, a small burr hole is drilled in the cranium and the composite is deposited into the cranium hole, where it is then driven intracranially to a specific location in the brain.

Magnetically-driven intracranial movement of the composites of the present invention may be carried out in accordance with U.S. Pat. No. 4,869,247 (Howard III”) and U.S. Pat. No. 6,216,030 (“Howard”), the specifications of which are incorporated by reference in their entirety.

We claim:

1. A method of treating brain injury comprising:
a) intrathecally administering a composite powder comprising superparamagnetic compound and a magnesium compound, and
b) magnetically transporting the composite into an injured brain.

2. The method of claim 1 wherein the superparamagnetic compound comprises iron oxide.

3. The method of claim 1 wherein the superparamagnetic compound comprises gadolinium.

4. The method of claim 1 wherein the magnesium compound is selected from the group consisting of magnesium metal, magnesium oxide, magnesium stearate, magnesium citrate, magnesium chloride, magnesium sulfate, magnesium carbonate, magnesium hydroxide, magnesium gluconate, magnesium phosphate and magnesium aspartate.

5. The method of claim 1 wherein the magnesium compound is magnesium oxide.

6. The method of claim 1 wherein the composite powder is in the form of magnesium oxide nanorods and iron oxide nanotubes.

7. The method of claim 6 wherein the composite powder comprises particulates having a mean aspect ratio of less than 30:1.

8. The method of claim 6 wherein the composite powder comprises particulates having a mean aspect ratio less than 10:1.

9. The method of claim 6 wherein the composite powder comprises particulates having a mean aspect ratio less than 3:1.

10. The method of claim 6 wherein the composite powder has a length of between 10 nm and 10 um.

11. The method of claim 6 wherein the composite powder has a length of between 30 nm and 1 um.

12. The method of claim 1 wherein the magnetic transport is carried out with external permanent magnets.

13. The method of claim 1 wherein the magnetic transport is carried out with at least one solenoid.

14. The method of claim 1 wherein the magnetic transport is carried out with a magnetic field array.

15. A superparamagnetic composite powder comprising;
a) a nanorod comprising a magnesium compound: and
b) a nanotube comprising an iron compound overlying the nanorod,
wherein the composite has an aspect ratio of less than 30:1.

16. The composite of claim 15 having an aspect ratio of less than 25:1.

17. The composite of claim 15 having an aspect ratio less than 10:1.

18. The composite of claim 15 having an aspect ratio less than 3:1.

19. The composite of claim 15 having a length of between 10 nm and 10 um.

20. The composite of claim 15 having a length of between 30 nm and 1 um.

21. A method of treating brain injury comprising:
a) intrathecally administering a composite powder comprising superparamagentic nanoparticle and a therapeutic agent compound, and
b) magnetically transporting the composite into an injured brain.

22. A method of treating stroke in a brain of a patient, comprising:
a) administering a composite powder comprising superparamagentic nanoparticle and an effective amount of a thiazole, and
b) imposing a magnetic field upon the brain.

23. The method of claim 22 wherein the administration of the composite is accomplished intravenously.

24. The method of claim 22 wherein the superparamagnetic compound comprises iron oxide.

25. The method of claim 22 wherein the superparamagnetic compound comprises gadolinium.

26. The method of claim 22 wherein the magnetic field is imposed for at least 1 hour.

27. The method of claim 22 wherein the magnetic field is imposed for at least 5 hours.

28. The method of claim 22 wherein. the magnetic field has a maximum strength within the brain of between 0.1 and 3 tesla.

29. The method of claim 22 wherein the magnetic field has a maximum strength within the brain of between 0.3 and 1 tesla.

30. The method of claim 22 wherein the magnetic field is imposed with external permanent magnets.

31. The method of claim 22 wherein the magnetic transport is imposed with at least one solenoid.

32. The method of claim 22 wherein the magnetic transport is imposed with a magnetic field array.

33. The method of claim 22 wherein the thiazole is a benzothiazole.

34. The method of claim 22 wherein the thiazole comprises lubeluzole.

35. A composite powder comprising superparamagentic nanoparticle and an effective amount of a thiazole.

36. The composite of claim 33 wherein the superparamagnetic compound comprises iron oxide.

37. The composite of claim 33 wherein the superparamagnetic compound comprises gadolinium.

38. The composite of claim 33 wherein the thiazole is a benzothiazole.

39. The composite of claim 33 wherein the thiazole comprises lubeluzole.

40. A method of treating brain injury comprising:
a) administering a composite powder comprising superparamagentic nanoparticle and a therapeutic agent compound into an injured brain, and
b) applying a magnetic field to the composite powder in the injured brain.
